(19) United States
(12) Patent Application Publication
(10) Pub. No.: US 2009/0062867 A1
(43) Pub. Date: Mar. 5, 2009

(54) Surgical retaining system

(75) Inventor: Joerg Schumacher, Teltow (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/231,335

(22) Filed: Aug. 28, 2008

(30) Foreign Application Priority Data
Aug. 30, 2007 (DE) 10 2007 042 958

(51) Int. Cl. A61B 17/04; A61B 17/56

(52) U.S. Cl. 606/308; 606/301; 606/305; 606/324; 606/60

(57) ABSTRACT

In order to simplify the producibility of a surgical retaining system comprising a head part and a threaded shaft which is mounted thereon so as to be pivotable, passes through an opening in the underside of the head part and has on its upper side a spherical thickened area which engages in an interior space of the head part, which is open upwards, and can be moved in the direction towards the underside of the interior space by a clamping device arranged on the head part and thereby fixed in position relative to the head part, it is suggested that the opening in the underside of the head part be of a cylindrical design and have an inner diameter which is slightly smaller than the outer diameter of the spherical thickened area so that the spherical thickened area, during insertion into the opening, is pressed into the cylindrical opening with the aid of the clamping device and is secured in the opening against any pivoting movement.

The invention relates to a surgical retaining system comprising a head part and a threaded shaft which is mounted thereon so as to be pivotable, passes through an opening in the underside of the head part and has, on its upper side, a spherical thickened area which engages in an interior space of the head, part, which is open upwards, and can be moved in the direction towards the underside of the interior space by a clamping device arranged on the head part and, as a result, can be fixed in position relative to the head part.

Surgical retaining systems of this type are known, for example, from U.S. Pat. No. 5,207,678 or DE 10 2005 021 879 A1. It is possible with retaining systems of this type to limit and to block the free pivotability of the spherical thickened area relative to the head part in that the head part is acted upon by the clamping device with a clamping force in the direction towards the underside of the interior space. This clamping force presses the spherical thickened area against an annular shoulder or a constriction or narrowing of the opening at the underside of the interior space so that the spherical thickened area is securely clamped as a result of abutment on this constriction or narrowing. This constriction or narrowing can have the shape of a spherical segment which is adapted to the spherical thickened area; it is also possible to use conical constrictions. In any case, relatively complicated geometries result, the production tolerances of which must be observed extremely exactly since, otherwise, the clamping effect is not ensured. In the case of, for example, spherical segments which are located internally, special measurement procedures must be developed in order to determine the exact course of the spherical annular surfaces; in the case of openings with a conical design, the conicality must be checked in a similar way. This involves considerable resources and, in addition, it is not, in practice, possible to avoid a high rejection quota occurring with such geometries.

The object of the invention is to design a generic surgical retaining system such that its production can be simplified.

This object is accomplished in accordance with the invention, in a surgical retaining system of the type described at the outset, in that the opening in the underside of the head part is of a cylindrical design and has an inner diameter which is slightly smaller than the outer diameter of the spherical thickened area so that the spherical thickened area, during insertion into the opening, is pressed into the cylindrical opening with the aid of the clamping device and is secured therein against any pivoting movement.

The production of a cylindrical opening is substantially easier than the formation of constrictions in the form of spherical segments or conical openings and it is also substantially easier to control whether such a cylindrical opening meets the necessary tolerances.

It has, surprisingly, been found that pressing the spherical thickened area into such a cylindrical opening leads to such a secure fit of the spherical thickened area in the cylindrical opening that the head part and the threaded shaft are secured against any pivoting in relation to one another in the desired manner. In this respect, it is not essential how deep the spherical thickened area is pushed into the cylindrical opening; it is essential only that the area of the greatest outer diameter of the spherical thickened area engages in the cylindrical opening.

The inner diameter of the opening can be between 0.005 mm and 0.5 mm smaller than the outer diameter of the spherical thickened area. When metals are used for the thickened area and for the wall material of the opening, this difference is relatively small and is preferably between 0.005 mm and 0.1 mm; when the spherical thickened area consists of a plastic material, greater differences can be used in the order of magnitude of between 0.05 mm and 0.5 mm. In any case, the difference is relatively slight and so the forces necessary to press the thickened area into the cylindrical opening are not too great.

In this respect, it is advantageous when the material of the spherical thickened area is plastically deformed when the spherical thickened area is pressed in so that the spherical thickened area is likewise deformed approximately cylindrically in the area of contact on the inner wall of the opening; as a result, a form-locking connection as a result of the abutment of a cylindrical ring of the thickened area on the cylindrical inner wall of the opening is also obtained in addition to pure clamping.

This effect can be augmented further in that the spherical thickened area is not designed as a uniform solid spherical surface but rather has a profiled surface or roughening, for example, as a result of circumferential grooves, as a result of circumferential ribs or the like. As a result of the irregular surface of the spherical thickened area, its plastic deformation is made easier when the spherical thickened area is pressed into the cylindrical opening and so an approximately cylindrical contour of the thickened area is achieved via a greater annular surface.

It may be provided for the head part to be slightly widenable elastically in the area of the opening in its underside so that it is easier to press the spherical thickened area in. The elastic widening should, in this respect, be very slight; it can, for example, be in the order of magnitude of one one-hundredth to several one-hundredths of a millimeter.

It is particularly advantageous when the clamping device has a stop, by means of which the insertion depth of the spherical thickened area in the opening of the underside is limited. This ensures that the spherical thickened area remains in the interior of the cylindrical opening even when the clamping device is actuated in full and will not be pushed out of this cylindrical opening downwards.

The clamping device can comprise, for example, a clamping screw which can be screwed into the interior space of the head part from above.

With such a configuration, it is advantageous when the stop is formed by the end of threads on the clamping screw and an internal thread on the head part accommodating them; these ends of the threads limit the screwing in of the clamping screw and, therefore, its insertion depth.

In a preferred embodiment it may be provided for a clamping element to be mounted in the interior space so as to be displaceable, this element being supported on the spherical thickened area of the threaded shaft and being displaceable in the direction towards the underside of the interior space by the clamping device. The clamping device therefore acts, in this case, indirectly on the spherical thickened area.

The clamping element is preferably designed as a cylindrical sleeve which abuts on the inner side of the interior space.

The clamping element can, itself, bear a stop which limits its insertion depth in the interior space of the head part. For example, such a stop could be formed by a snap-in element which engages in an opening in the inner wall of the interior space of the head part. Such a snap-in element can, at the same time, also secure the clamping element against any unintentional withdrawal out of the interior space.

In a preferred embodiment, it is provided, in addition, for the clamping element to have a contact surface for a retaining bar which is arranged transversely to the longitudinal direction of the interior space and projects through openings in the wall of the head part which are located opposite one another and for the clamping device to come to rest on the retaining bar during its advancement in the direction towards the underside of the head part and then to press the clamping element against the spherical thickened area via the retaining bar.

In this respect, it is favorable when the edge of the openings in the wall of the head part forms a stop for the retaining bar which limits the displacement of the retaining bar in the direction towards the underside of the interior space.

When the clamping device is actuated, the spherical thickened area of the threaded shaft is, in such a configuration, pushed downwards first of all via the retaining bar and the clamping element until it engages in the cylindrical opening in the underside of the interior space and so, as a result, the threaded shaft is secured against any pivoting in relation to the head part. As long as the retaining bar is not pressed against the edge of the openings in the wall of the head part, the pressing force on the retaining bar is still not large enough to secure this completely; this means that it remains displaceable in relation to the clamping element and so, in this position, the possibility of pivoting the threaded shaft in relation to the head part is ruled out but the retaining bar is displaceable. The retaining bar will not be permanently fixed in relation to the head part until the clamping element is actuated completely and the retaining bar pressed onto the edge of the openings in the wall of the head part. In this respect, the spherical thickened area of the threaded shaft will be moved further in the cylindrical opening but this does not alter anything with regard to the press fit in the cylindrical opening.

It is advantageous when the inner diameter of the interior space is slightly greater than the outer diameter of the spherical thickened area so that the spherical thickened area is freely displaceable in the interior space and can be advanced only by the clamping device owing to the application of pressing forces once it enters the cylindrical opening.

The following description of preferred embodiments of the invention serves to explain the invention in greater detail in conjunction with the drawings. These show:

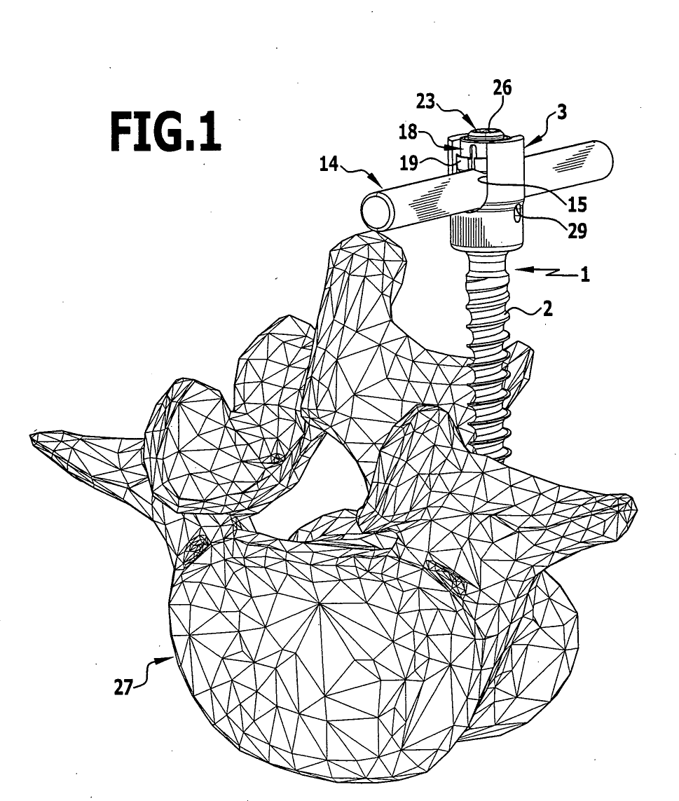

FIG. 1: a perspective view of an orthopedic retaining system on a vertebral bone;

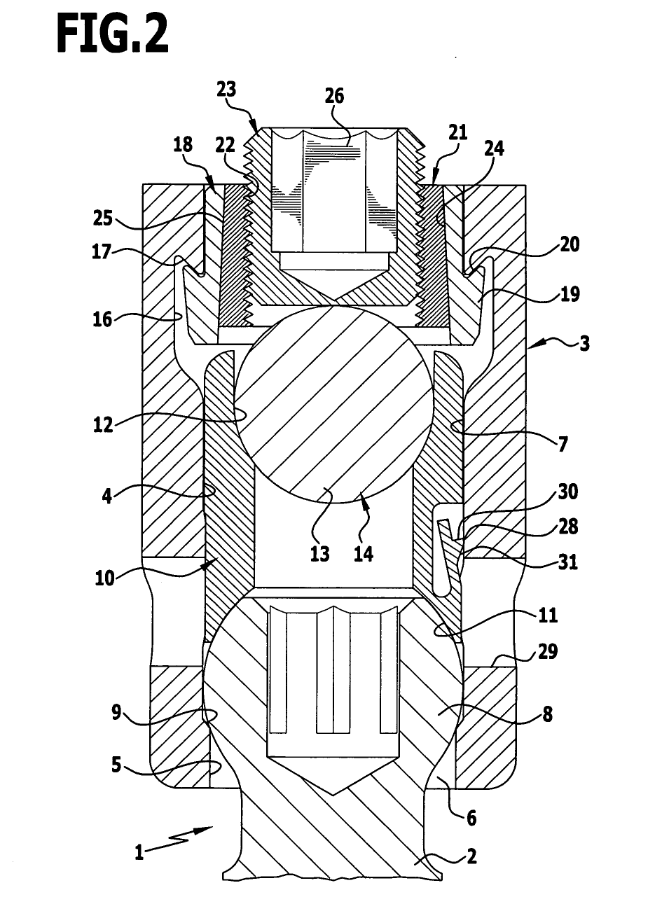

FIG. 2: a longitudinal sectional view through the head part of the orthopedic retaining system of FIG. 1 prior to the spherical thickened area being pushed into the cylindrical opening in the underside of the head part and

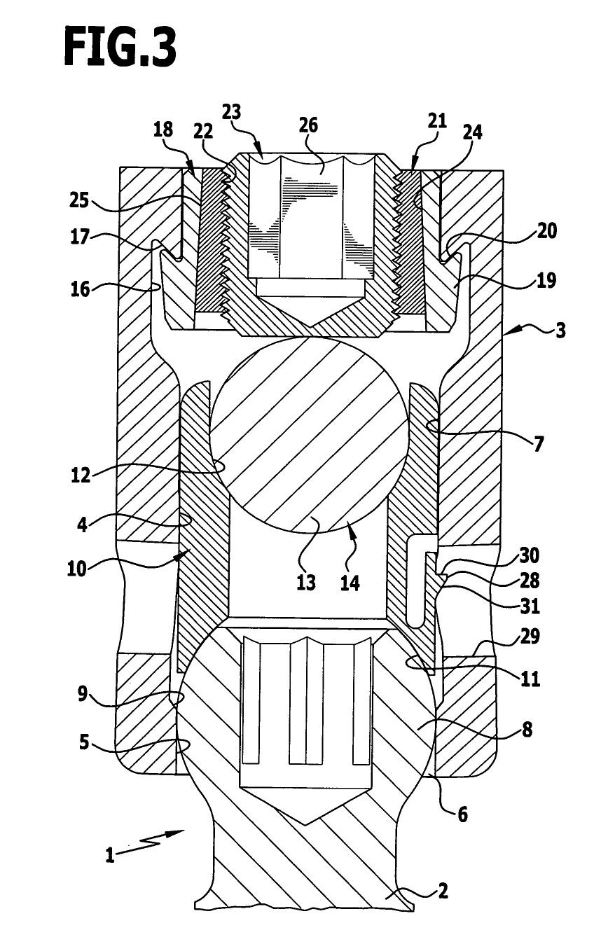

FIG. 3: a view similar to FIG. 2 after the spherical thickened area has been pushed into the cylindrical opening in the underside of the head part.

The orthopedic retaining system illustrated in the drawings comprises a bone screw 1 with a threaded shaft 2 which is preferably designed to be self-cutting and a head part 3. The head part 3 has the shape of a cylindrical sleeve with a continuous interior space 4, the inner diameter of which is the same over its entire height but is slightly smaller in the area of the lower end of the head part 3 owing to the formation of a cylindrical opening 5. This is illustrated in the illustrations of FIGS. 2 and 3 by a step 9 which projects inwardly and, for the sake of clarification, is illustrated as projecting inwardly to a greater extent than is actually the case.

The opening 5 has a circular cylindrical inner wall 6 which has a slightly smaller outer diameter in relation to the inner wall 7 of the interior space 4 and extends concentrically to this inner wall 7.

The bone screw 1 is inserted into the interior space 4 from above with its threaded shaft 2 and passes through the opening 5. At its upper end, the threaded shaft 2 has a spherical thickened area 8, the outer diameter of which is slightly smaller than the inner diameter of the inner wall 7 of the interior space 4 but slightly greater than the inner diameter of the inner wall 6 of the opening 5. For example, the outer diameter of the spherical thickened area 8 can be between 0.005 mm and 0.5 mm greater than the inner diameter of the inner wall 6 of the opening 5 and the inner diameter of the inner wall 7 of the interior space 4 can be 0.001 mm to 2 mm greater than the outer diameter of the spherical thickened area 8. In this respect, the differences in measurement between the outer diameter of the spherical thickened area 8 and the inner diameter of the inner wall 6 are also dependent, in particular, on the material which is used for the spherical thickened area 8 and for the inner wall 6. When both components are of a metallic configuration, the difference in measurement is preferably in the order of magnitude of between 0.005 mm and 0.1 mm; when the spherical thickened area 8 consists of a plastic material, the difference can be greater and is then preferably between 0.05 mm and 0.5 mm.

In the embodiment illustrated in the drawings, the spherical thickened area has a smooth, solid spherical surface. In a modified embodiment not illustrated in the drawings, this surface is, however, profiled or roughened either by a regular structure, for example, by circumferential grooves and circumferential ribs or by a chemical or mechanical roughening and so areas result, the outer diameter of which, i.e., their distance from the central point of the spherical thickened area differs. This makes a plastic deformation at the surface of the spherical thickened area 8 easier when this spherical thickened area 8 is pressed into the cylindrical opening 5 and so, with this plastic deformation, the spherical thickened area 8 is fixed in position not only by means of a pure press fit but also, to a slight extent, by a form-locking connection which results by way of the cylindrical deformation at the contact area of the spherical thickened area 8 on the inner wall 6 of the opening 5.

During insertion of the threaded shaft 2, the spherical thickened area 8 can easily be advanced as far as the lower end of the interior space 4 and rests first of all on the step 9. In this position, the threaded shaft 2 is pivotable in all directions in relation to the head part 3; a polyaxial mounting is, therefore, obtained.

A clamping sleeve 10 is inserted into the interior space 4 from above, above the spherical thickened area 8, and this sleeve abuts with its outer side areally on the inner wall 7 of the interior space 4 and is freely displaceable in it in a longitudinal direction. It is supported with its lower edge 11 on the upper side of the spherical thickened area 8; in addition, the lower edge 11 is designed in the shape of a spherical segment so as to correspond to the spherical contour of the spherical thickened area 8.

On its upper side, the clamping sleeve 10 has two U-shaped openings 12 which are located opposite one another, are open upwards and form a supporting surface 13 for a cylindrical retaining bar 14 which rests on the supporting surfaces 13 transversely to the longitudinal direction of the clamping sleeve 10 and projecting to both sides though recesses 15 in the wall of the head part 3.

A circumferential groove 16 is integrally formed in the inner wall 7 of the interior space 4 next to the upper end of the head part 3, the upper side wall 17 of this groove being designed to slope upwards at an angle from the inside to the outside. A retaining ring 18, which is inserted into the interior space 4 from above, engages in this circumferential groove 16 with projections 19 which project radially outwards and abut, for their part, on the side surface 17 of the circumferential groove 16 via a side surface 20 sloping upwards at an angle. The retaining ring 18 is, as a result, secured against any withdrawal out of the interior space 4; it can, however, be pushed into the interior space 4 from above owing to elastic widening of the oppositely located sides of the head part 3; in this respect, the projections 19 slide along the inner wall of the widened interior space 4 until they enter the circumferential groove 16.

The retaining ring 18 surrounds a screw sleeve 21 with an internal thread 22, into which a clamping screw 23 is screwed. This has an internal polyhedron 24 for the insertion of a rotary instrument, with the aid of which the clamping screw 23 can be screwed into the internal thread 22 to a greater or lesser depth and thereby abuts on the upper side of a retaining bar 14 placed on the supporting surfaces 13.

The inner surface 24 of the retaining ring 18 and the outer surface 25 of the screw sleeve 21 are designed to be slightly conical in the same way with a diameter which decreases upwards and so a clamping effect is achieved when the screw sleeve 21 is moved in relation to the retaining ring 18; on account of the small angle of conicality this effect is a self-locking clamping effect.

When the surgical retaining system described is used, the threaded shaft 2 is pushed, first of all, into the head part 3 and the threaded shaft 2 is screwed into a bone, for example, into a vertebral bone 27, as illustrated in FIG. 1, by means of a suitable rotary tool which can be inserted into an internal polyhedron 26 in the spherical thickened area 8. In this respect, the threaded shaft 2 is readily and freely rotatable in relation to the head part 3. Subsequently, the clamping sleeve 10 and a constructional unit which consists of the retaining ring 18, the screw sleeve 21 and the clamping screw 23 are pushed into the interior space 4 of the head part 3. For this purpose, the clamping screw 23 is, of course, not yet screwed deep into the screw sleeve 21 and so it is possible to insert this unit.

A retaining bar 14 can be placed inside or pushed in either prior to the insertion of this unit or also thereafter. Before the clamping screw 23 is securely screwed in, the threaded shaft 2 can be pivoted in all directions in relation to the head part 3 and also the retaining bar 14 can be displaced in a longitudinal direction and turned about its longitudinal axis.

When the clamping screw 23 is screwed in, it abuts on the upper side of the retaining bar 14 and presses this, together with the clamping sleeve 10, downwards against the spherical thickened area 8. In this respect, this is advanced past the step 9 into the cylindrical opening 5, as illustrated in FIG. 3. As soon as the spherical thickened area 8 has entered the opening 5 with its greatest outer diameter, a press fit results due to the overdimensional tolerance, i.e., due to the slightly greater outer diameter of the spherical thickened area 8 in relation to the inner diameter of the opening 5 and the pivoting of the threaded shaft 2 is terminated by this press fit. As is apparent in the illustration of FIG. 3, the clamping sleeve 10 is inserted into the interior space 4 to such a depth that a snap-in nose 28, which is arranged on the clamping sleeve 10 and projects radially beyond its outer surface, enters an opening 29 in the inner wall 7 of the interior space 4. The snap-in nose 28 is arranged on the clamping sleeve 10 so as to be elastically displaceable radially inwards and can be moved radially inwards to such an extent that it slides past the inner wall of the interior space 4 during the displacement of the clamping sleeve 10. The snap-in nose 28 has a flat, upper snap-in surface 30 and an inclined lower side surface 31 acting as a slide-on surface. As a result of the snap-in nose 28 engaging in the opening 29, the clamping sleeve 10 is secured against any withdrawal out of the interior space 4; during insertion, the snap-in nose is moved radially inwards as a result of the lower side surface 31 acting as a slide-on surface and can slide along the inner wall of the interior space 4 until it enters the opening 29.

The clamping sleeve 10 can be designed such that its insertion depth in the interior space 4 is limited. This could be brought about, for example, by the snap-in nose 28 when this strikes the lower end of the opening 29. This is not illustrated in the drawings but a corresponding modification would easily be possible. The clamping sleeve could also have, as stop, a collar which strikes the lower edge of the opening 29 or a similar projection which interacts with the inner wall 7 of the interior space 4.

When the clamping screw 23 is screwed in to such an extent that the spherical thickened area 8 is located in the cylindrical opening 5, the pivotability of the threaded shaft 2 is terminated but the displaceability of the retaining bar 14 is not yet completely terminated since the retaining bar is still displaceable relative to the clamping sleeve 10 despite the clamping forces acting on it. This can also be aided by the fact that the clamping sleeve 10 consists of a plastic material which has low friction and, therefore, aids the displacement of the retaining bar 14.

When, on the other hand, the clamping screw 23 is screwed in further, the retaining bar 14 finally abuts on the lower edge of the recess 15 in the wall of the head part 2 and so a firm tensioning of the retaining bar 14 occurs. As a result, the retaining bar is secured against any longitudinal displacement and any rotation; in addition, the insertion depth of the spherical thickened area 8 in the cylindrical opening 5 is limited, as a result, and so there is no risk of the spherical thickened area 8 being able to be pushed downwards out of the cylindrical opening 5 due to the clamping screw 23 being turned in to too great an extent.

The clamping device described and illustrated in the drawings comprises a conical clamping by means of the conical configuration of the inner and outer surfaces, respectively, of the retaining ring 18 and the screw sleeve 21. Instead, other clamping devices could also be used, for example, a simple clamping screw which is screwed into an internal thread of the interior space or a nut which is screwed onto an external thread of the head part 3 and supported on the retaining bar 14. The conical clamping described is merely represented and discussed as one example of such a clamping device.

Metals which are biocompatible are considered essentially as materials for the parts described, for example, titanium or titanium alloys; in the case of the clamping sleeve 10, pure titanium is preferably used. The spherical thickened area can likewise consist of titanium or a titanium alloy but is it also possible to use a plastic material for the spherical thickened area, for example, polyether ether ketone or similar, biocompatible plastic materials. These plastic materials have the advantage that they are plastically deformed to a greater extent when the spherical thickened area 8 is pressed into the opening 5 and so a distinct form-locking fixing can also take place in addition to the clamping effect.

1-13. (canceled)

14. Surgical retaining system comprising a head part and a threaded shaft mounted thereon so as to be pivotable, said shaft passing through an opening in the underside of the head part and having on its upper side a spherical thickened area engaging in an upwardly open interior space of the head part and being able to be moved in the direction towards the underside of the interior space by means of a clamping device arranged on the head part and thereby to be fixed in position relative to the head part, wherein the opening in the underside of the head part is of a cylindrical design and has an inner diameter slightly smaller than the outer diameter of the spherical thickened area so that the spherical thickened area, during insertion into the opening, is pressed into the cylindrical opening with the aid of the clamping device and is secured in said opening against any pivoting movement.

15. Surgical retaining system as defined in claim 14, wherein the inner diameter of the opening is between 0.005 mm and 0.1 mm smaller than the outer diameter of the spherical thickened area.

16. Surgical retaining system as defined in claim 14, wherein the head part is adapted to be slightly widened elastically at its underside in the area of the opening.

17. Surgical retaining system as defined in claim 14, wherein the clamping device has a stop for limiting the insertion depth of the spherical thickened area in the opening in the underside of the head part.

18. Surgical retaining system as defined in claim 14, wherein the clamping device comprises a clamping screw adapted to be screwed into the interior space of the head part from above.

19. Surgical retaining system as defined in claim 17, wherein the stop is formed by the end of threads on the clamping screw and an internal thread on the head part accommodating them.

20. Surgical retaining system as defined in claim 14, wherein a clamping element is mounted for displacement in the interior space, said element being supported on the spherical thickened area of the threaded shaft and displaceable by the clamping device in the direction towards the underside of the interior space.

21. Surgical retaining system as defined in claim 20, wherein the clamping element is designed as a cylindrical sleeve abutting on the inner side of the interior space.

22. Surgical retaining system as defined in claim 20, wherein the clamping element has a contact surface for a retaining bar arranged transversely to the longitudinal direction of the interior space and projecting through openings in the wall of the head part located opposite one another and wherein the clamping device comes to rest on the retaining bar when advancing in the direction towards the underside of the head part and then presses the clamping element against the spherical thickened area via the retaining bar.

23. Surgical retaining system as defined in claim 22, wherein the edge of the openings in the wall of the head part forms a stop for the retaining bar limiting the displacement of the retaining bar in the direction towards the underside of the interior space.

24. Surgical retaining system as defined in claim 22, wherein the clamping element has a stop limiting the depth of insertion of the clamping element in the interior space.

25. Surgical retaining system as defined in claim 14, wherein the inner diameter of the interior space is slightly greater than the outer diameter of the spherical thickened area.

26. Surgical retaining system as defined in claim 14, wherein the spherical thickened area is profiled or roughened on its surface.
